(19) United States
(12) Patent Application Publication
(10) Pub. No.: US 2003/0115619 A1
(43) Pub. Date: Jun. 19, 2003

(54) Dnmt3L-related cloning and reproductive methods

(76) Inventors: Timothy H. Bestor, New York, NY (US); Deborah Bourc'his, New York, NY (US)

Correspondence Address: Cooper and Dunham LLP, 1185 Avenue of the Americas, New York, NY, 10036, US

(21) Appl. No.: 10/301,041

(22) Filed: Nov. 21, 2002

Related U.S. Application Data
(60) Provisional application No. 60/332,081, filed on Nov. 21, 2001.

(51) Int. Cl. A01K 67/027; C12N 5/08; C12N 15/85

(52) U.S. Cl. 800/14; 800/15; 800/16; 800/17; 800/18; 435/325; 435/320.1; 435/366; 435/455

(57) ABSTRACT

This invention provides methods for reducing the amount of Dnmt3L in a cell, obtaining a cell having a reduced amount of Dnmt3L, and cells, tissues, organs and non-human transgenic mammals having a reduced amount of Dnmt3L. This invention further provides related methods for producing a mammalian zygote in vitro which, upon successful subsequent development in utero, gives rise to a mammal having a reduced susceptibility to an abnormality associated with epigenetic instability. This invention further provides methods for determining the amount of Dnmt3L in a cell, and methods for determining whether agents bind to Dnmt3L or decrease the amount of Dnmt3L in a cell. Finally, this invention provides related nucleic acids and compositions.

This application claims priority of provisional application U.S. Serial No. 60/332,081, filed Nov. 21, 2001, the contents of which are incorporated herein by reference.

Throughout this application, various references are cited. Disclosure of these references in their entirety is hereby incorporated by reference into this application to more fully describe the state of the art to which this invention pertains.

The invention described herein was made with government support under NIH Grants GM59377 and HD37687. Accordingly, the United States government has certain rights in this invention.

### BACKGROUND OF THE INVENTION

Complementary sets of genes are epigenetically silenced in male and female gametes in a process termed genomic imprinting. Genomic imprinting imposes a requirement for biparental reproduction. Uniparental mammalian conceptuses are inviable (1-3), and lack of imprinting of specific chromosomes or chromosome segments due to uniparental isodisomy or deletions of imprinting centers causes distinct developmental defects according to the chromosomal region involved (4,5). The poor success rate and unpredictable phenotypic variation seen in mammals produced by cloning procedures is also likely to involve disturbances of genomic imprints (6). However, very little is known of the mechanisms that establish and maintain genomic imprints.

The Dnmt3L gene is expressed during gametogenesis at stages where genomic imprints are established. Targeted disruption of Dnmt3L caused azoospermia in homozygous males and heterozygous progeny of homozygous females died prior to midgestation. Bisulfite genomic sequencing of DNA from oocytes and embryos showed that removal of Dnmt3L prevented methylation of sequences that are normally maternally methylated. The defect was specific to imprinted regions and global genome methylation levels were not affected. Lack of maternal methylation imprints in heterozygous embryos derived from homozygous mutant oocytes caused biallelic expression of genes that are normally expressed only from the allele of paternal origin.

To date, the proteins responsible for regulating DNA imprinting have not been identified.

### SUMMARY OF THE INVENTION

This invention provides a method for reducing the amount of Dnmt3L in a cell comprising contacting the cell under suitable conditions with an agent that specifically inhibits the expression of Dnmt3L in the cell, thereby reducing the amount of Dnmt3L in the cell.

This invention also provides a method for obtaining a cell having a reduced amount of Dnmt3L comprising isolating a cell from a transgenic non-human mammal whose cells contain a reduced amount of Dnmt3L.

This invention further provides an isolated cell having a reduced amount of Dnmt3L, an isolated population of cells having a reduced amount of Dnmt3L, a tissue comprising cells having a reduced amount of Dnmt3L, an organ comprising cells having a reduced amount of Dnmt3L, and a non-human transgenic mammal comprising cells having a reduced amount of Dnmt3L.

This invention further provides a method for producing a mammalian zygote in vitro which, upon successful subsequent development in utero, gives rise to a mammal having a reduced susceptibility to an abnormality associated with epigenetic instability, comprising contacting a mammalian sperm cell and a mammalian ovum in vitro under conditions permitting fertilization of the ovum by the sperm, wherein the sperm cell, the ovum or both are contacted with an agent that reduces the amount of Dnmt3L therein, thereby producing the mammalian zygote.

This invention further provides a method for producing a

Dnmt3L⁻ non-human mammalian cell, which method comprises introducing a non-human mammalian Dnmt3L⁻ nucleus into a non-human mammalian enucleated oocyte or zygote under suitable conditions, thereby producing a Dnmt3L⁻ cell.

This invention further provides a method for producing a non-human mammalian zygote which, upon successful subsequent development in utero, gives rise to a non-human mammal having a reduced susceptibility to an abnormality associated with epigenetic instability, comprising introducing a non-human mammalian Dnmt3L nucleus into a non-human mammalian enucleated oocyte or zygote under suitable conditions, thereby producing the zygote.

This invention provides a first method for determining the amount of Dnmt3L in a cell comprising (a) contacting the cell's contents with an agent that specifically forms a complex with Dnmt3L, (b) determining the amount of complex formed in step (a), and (c) comparing the amount of complex so determined with a known standard, thereby determining the amount of Dnmt3L in the cell.

This invention also provides a second method for determining the amount of Dnmt3L in a cell comprising (a) determining the amount of Dnmt3L-encoding mRNA in the cell and (b) comparing the amount so determined with a known standard permitting the quantitation of Dnmt3L based on a known amount of Dnmt3L-encoding mRNA, thereby determining the amount of Dnmt3L in the cell.

This invention further provides a method for determining whether an agent binds to Dnmt3L comprising contacting the agent with Dnmt3L under suitable conditions, and determining whether the agent forms a complex with the Dnmt3L, the formation of a complex indicating that the agent binds to Dnmt3L.

This invention provides a first method for determining whether an agent decreases the amount of Dnmt3L in a cell, which method comprises the steps of:

(a) contacting the cell with the agent under suitable conditions;

(b) determining the amount of Dnmt3L in the cell after a suitable period of time; and

(c) comparing the amount of Dnmt3L determined in step (b) with the amount of Dnmt3L in a cell in the absence of the agent, a lower amount of Dnmt3L in the cell contacted with the agent indicating that the agent decreases the amount of Dnmt3L in the cell.

This invention also provides a second method for determining whether an agent decreases the expression of Dnmt3L in a cell, which method comprises the steps of:

(a) contacting the cell with the agent under suitable conditions;

(b) determining the amount of Dnmt3L expression in the cell after a suitable period of time; and

(c) comparing the amount of Dnmt3L expression determined in step (b) with the amount of Dnmt3L expression in a cell in the absence of the agent, a lower amount of Dnmt3L expression in the cell contacted with the agent indicating that the agent decreases the amount of Dnmt3L expression in the cell.

This invention further provides an isolated nucleic acid that specifically hybridizes to Dnmt3L-encoding mRNA. Finally, this invention provides a composition of matter comprising the instant nucleic acid and a carrier.

### BRIEF DESCRIPTION OF THE FIGURES

FIG. 1

Relationship of Dnmt3L to other mammalian DNA methyltransferases and disruption of Dnmt3L gene. (A) Sequence relationships among mammalian DNA methyltransferases. Catalytic motifs are designated with roman numerals. Motifs are absent from Dnmt3L while the cysteine-rich regions and other sequences show strong similarities with Dnmt3A and Dnmt3B. At right is a ClustalW representation of sequence similarities within the region spanning catalytic motifs I-VIII. The corresponding region of Dnmt3L was identified by alignment with Dnmt3A and Dnmt3B. (B) Disruption of the Dnmt3L gene by homologous recombination in ES cells. Methods were as described (25,27), except that CSL3 ES cells derived from blastocysts of strain 129SvEv/Tac were used. The disruption brings the b-geo reporter/resistance gene under the control of the endogenous Dnmt3L promoter. The X 3′ of exon 1 indicates site of 3 in-frame stop codons; the polyadenylation signal prevents expression of downstream exons. Restriction endonuclease sites are: B, BamHI; H, HindIII; Xh, XhoI; Xb, XbaI. DNA blot hybridization (C) after cleavage of ES cell DNA with BamHI confirmed that the expected homologous recombination event had taken place. The mutant allele was designated Dnmt3L^{G}.

FIG. 2

Expression of Dnmt3L gene in male and female germ cells and sterility of homozygous Dnmt3L^{G} males. (A) Accumulation of β-geo reporter under control of Dnmt3L promoter specifically in growing oocytes as assessed by staining adult ovaries with X-Gal. Growing oocytes at all stages are stained, but primary oocytes and somatic cells are unstained. Oviduct is at bottom. (B) Transcription from DnmL3L promoter in seminiferous tubules of fetal testis. Staining for β-geo with X-Gal was as in (A). Wild type testis from 17.5 dpc mouse fetus is at left; testis from heterozygous littermate is at right. Staining within seminiferous tubules was present in prospermatogonia. Postpartum and adult testes showed much less b-gal activity. (C) Hypogonadism in homozygous Dnmt3L^{G} testes. Testis of wild type litter mate is at left. (D) Sertoli-cell-only phenotype in seminiferous tubules of homozygous Dnmt3L^{G} adult males. Tubule lumen is occupied only by cytoplasmic processes of Sertoli cells. (E) Section of seminiferous tubule from wild type littermate.

FIG. 3

Maternal effect lethal phenotype in heterozygous embryos derived from homozygous Dnmt3L^{G} females. (A) Exencephalic embryo at 9.5 dpc. (B) Developmental failure of heterozygous progeny of homozygous Dnmt3L^{G} females when transferred to uteri of wild type females. Left uterine horn received 15 mutant embryos; right horn received 15 wildtype control embryos. At 13.5 dpc mutant conceptuses are represented only by necrotic implantation sites (blue marks at left) while development of wildtype conceptuses (white marks) in right horn is normal. (C) Normal chorio-allantoic fusion in a wild type concetus at 8.5 dpc. (D) Abnormalities of extraembryonic structures and failure of chorio-allantoic fusion in heterozygous Dnmt3L^{G} 9.5 dpc embryo derived from homozygous oocyte. Note abnormal separation of allantois (All) and chorion (Chor) by comparison with (C), thickened chorion, hyperproliferation of secondary trophoblastic giant cells in ectoplacental cone (EPC) and of yolk sac endoderm (lateral to allantois). Excess maternal blood apposed to ectoplacental cone is also apparent.

FIG. 4

Lack of maternal methylation imprints in homozygous Dnmt3L^{G} oocytes and in heterozygous embryos derived from them. (A) Methylation of all CpG dinucleotides in the DMR of Snrpn in control embryos and light and variable methylation of the same sequence in homozygous Dnmt3L^{G} oocytes. (B) Lanes 1 and 2, lack of methylation at HhaI sites in the DMR of the maternal allele of Snrpn in heterozygous embryos derived from homozygous Dnmt3L^{G} oocytes. DNA was digested with HhaI and NdeI. Lanes 36, global genome methylation was not decreased in heterozygous embryos derived from homozygous Dnmt3L^{G} oocytes as assessed by sensitivity to HpaII (lanes 4 and 6). Lanes 3 and 5 contained MspI digests. Lanes 7-10, IAP retroposons and pericentric satellite DNA are not demethylated in heterozygous embryos derived from homozygous Dnmt3L^{G} oocytes. In all cases DNA was purified from embryos at 8.5 dpc. Samples marked G/+ were from heterozygous progeny of homozygous Dnmt3L^{G} females. (C) Loss of allele-specific methylation at the maternally imprinted genes Snrpn and Peg1 with normal monoallelic methylation at the paternally imprinted H19 gene. Controls show monoallelic methylation for all tested sequences (upper portion of (C)).

FIG. 5

Biallelic expression of maternally imprinted genes in heterozygous progeny of homozygous Dnmt3L^{G} females. Expressed polymorphisms were introduced by crossing homozygous Dnmt3L^{G} females to M. m. castaneus males or to males containing M. m. castaneus chromosome 7 on a C57BL/6J strain background. Regions containing the polymorphisms were recovered by RT-PCR and the allele-specific expression determined by direct sequencing of the products in (A) or by agarose gel electrophoresis in (B). (A) Snrpn, Necdin, Zfp127, KcnqloLl, and Peg3 were expressed only from the paternal allele in control embryos (top row) but were expressed from both alleles in heterozygous progeny of homozygous Dnmt3L^{G} females (bottom row). H19 remained imprinted and was expressed only from the maternal allele, as predicted from the methylation data of FIG. 4. (B) Expression of Igf2 remained monoallelic and paternal, in agreement with the retention of imprinted expression of H19 and the reciprocal imprinting of Igf2 and H19 (4). The Cdkn1 and Ipl genes were silenced biallelically as a result of reactivation of maternal Kcnqlot1 transcription (22-24). Polymorphisms are described in the literature. Samples marked G/+ were from heterozygous progeny of homozygous Dnmt3L^{G} females.

### DETAILED DESCRIPTION OF THE INVENTION

Definitions

As used in this application, except as otherwise expressly provided herein, each of the following terms shall have the meaning set forth below.

“Antibody” shall include, by way of example, both naturally occurring and non-naturally occurring antibodies. Specifically, this term includes polyclonal and monoclonal antibodies, and fragments thereof. Furthermore, this term includes chimeric antibodies and wholly synthetic antibodies, and fragments thereof.

“Anti-sense nucleic acid” shall mean any nucleic acid (e.g., RNA) which, when introduced into a cell, specifically hybridizes to at least a portion of an mRNA in the cell encoding a protein (“target protein”) whose expression is to be inhibited, and thereby inhibits the target protein's expression.

“Carriers” are well known to those skilled in the art and include, but are not limited to, 0.01-0.1 M and preferably 0.05 M phosphate buffer or 0.8% saline. Additionally, such pharmaceutically acceptable carriers can be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions and suspensions, including saline and buffered media. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like.

“Catalytic nucleic acid” shall mean a nucleic acid that specifically recognizes a distinct substrate and catalyzes the chemical modification of this substrate.

“DNAzyme” shall mean a catalytic nucleic acid that is DNA or whose catalytic component is DNA, and which specifically recognizes and cleaves a distinct target nucleic acid sequence, which can be either DNA or RNA. Each DNAzyme has a catalytic component (also referred to as a “catalytic domain”) and a target sequence-binding component consisting of two binding domains, one on either side of the catalytic domain.

“Expressible nucleic acid” shall mean a nucleic acid encoding a nucleic acid of interest and/or a protein of interest, which nucleic acid is an expression vector, plasmid or other construct which, when placed in a cell, permits the expression of the nucleic acid or protein of interest. Expression vectors and plasmids are well known in the art.

“Inhibiting” the expression of a gene in a cell shall mean either lessening the degree to which the gene is expressed, or preventing such expression entirely.

As used herein, “mammal” includes, but is not limited to, a mouse, a rat, a dog, a cat, a cow, a sheep, a pig, a guinea pig, a ferret, a rabbit, and a primate. In the preferred embodiment, the mammal is a human.

“Nucleic acid” shall mean any nucleic acid molecule, including, without limitation, DNA, RNA and hybrids thereof. The nucleic acid bases that form nucleic acid molecules can be the bases A, C, G, T and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, N.J., USA).

“Ribozyme” shall mean a catalytic nucleic acid molecule which is RNA or whose catalytic component is RNA, and which specifically recognizes and cleaves a distinct target nucleic acid sequence, which can be either DNA or RNA. Each ribozyme has a catalytic component (also referred to as a “catalytic domain”) and a target sequence-binding component consisting of two binding domains, one on either side of the catalytic domain.

“Specifically hybridize” to a nucleic acid shall mean, with respect to a first nucleic acid, that the first nucleic acid hybridizes to a second nucleic acid with greater affinity than to any other nucleic acid.

“Specifically inhibit” the expression of a protein shall mean to inhibit that protein's expression (a) more than the expression of any other protein, or (b) more than the expression of all but 10 or fewer other proteins.

“Suitable conditions” shall have a meaning dependent on the context in which this term is used. That is, when used in connection with an antibody, for example, the term shall mean conditions that permit an antibody to bind to its corresponding antigen. When this term is used in connection with nucleic acid hybridization, the term shall mean conditions that permit a nucleic acid of at least 15 nucleotides in length to hybridize to a nucleic acid having a sequence complementary thereto. When used in connection with contacting an agent to a cell, this term shall mean conditions that permit an agent capable of doing so to enter a cell and perform its intended function. In one embodiment, the term “suitable conditions” as used herein means physiological conditions.

Embodiments of the Invention

This invention is based on the surprising discovery that Dnmt3L plays a role in regulating the DNA imprinting process. This invention therefore has importance, in particular, for reducing birth defects and certain other disorders observed in humans and animals resulting from in vitro fertilization and cloning, respectively.

Specifically, this invention provides a method for reducing the amount of Dnmt3L in a cell comprising contacting the cell under suitable conditions with an agent that specifically inhibits the expression of Dnmt3L in the cell, thereby reducing the amount of Dnmt3L in the cell.

In one embodiment of the invention, the agent is a nucleic acid, which can be, for example, DNA or RNA. Nucleic acids useful as agents in this regard include, without limitation, RNAi molecules, anti-sense nucleic acids, ribozymes and DNAzymes. Methods for designing such agents based on the sequence of a target nucleic acid sequence are well known.

In other embodiments, the cell is, preferably, a human cell, and can also be a stem cell (e.g., CD34⁺ cell), an embryonic cell (e.g., embryonic stem cell), and a gamete (i.e., sperm cell or ovum). Preferably, the expression of Dnmt3L in the cell is eliminated through, e.g., removal of or interference with expression of the Dnmt3L gene.

This invention also provides a method for obtaining a cell having a reduced amount of Dnmt3L comprising isolating a cell from a transgenic non-human mammal whose cells contain a reduced amount of Dnmt3L. The cell is, preferably, a human cell, and can also be a stem cell, an embryonic cell (e.g., embryonic stem cell), and a gamete (i.e., sperm cell or ovum). Preferably, the expression of Dnmt3L in the cells is eliminated, or at least, the cells contain no Dnmt3L.

This invention further provides an isolated cell having a reduced amount of Dnmt3L, and preferably no Dnmt3L. The cell can be of any type set forth above, and can also be, for example, a hepatocyte, a neuron, a cardiac cell, a stem cell, an embryonic cell and a skin cell.

This invention further provides an isolated population of cells having a reduced amount of Dnmt3L, and preferably no Dnmt3L. The population of cells can be of any type set forth above, and can also be, for example, hepatocytes, neurons, cardiac cells, stem cells, embryonic cells and skin cells.

This invention further provides a tissue comprising cells having a reduced amount of Dnmt3L, and preferably no Dnmt3L. The tissue can be of any type, and is preferably tissue useful for purposes of xenogenic grafting and stem cell-based regeneration. Such tissues include, for example, hepatic tissue, neuronal tissue, cardiac tissue and dermal tissue.

This invention further provides an organ comprising cells having a reduced amount of Dnmt3L, and preferably no Dnmt3L. The organ can be of any type, and is preferably an organ useful for purposes of xenogenic transplant and stem cell-based regeneration. Such organs include, for example, a liver, a heart and skin.

This invention further provides a non-human transgenic mammal comprising cells having a reduced amount of Dnmt3L, and preferably no Dnmt3L. In one embodiment, the mammal is a mouse, a rat, a hamster, a pig, a cow, a sheep, a non-human primate, a cat or a dog.

This invention further provides a method for producing a mammalian zygote in vitro which, upon successful subsequent development in utero, gives rise to a mammal having a reduced susceptibility to an abnormality associated with epigenetic instability, comprising contacting a mammalian sperm cell and a mammalian ovum in vitro under conditions permitting fertilization of the ovum by the sperm, wherein the sperm cell, the ovum or both are contacted with an agent that reduces the amount of Dnmt3L therein, thereby producing the mammalian zygote. As used herein, “epigenetic instability” means the tendency of a cell's DNA imprinting status to change. This method has particular advantages in reducing the incidence of birth defects and enlarged placentas associated with in vitro fertilization, especially in humans.

In this method, the sperm cell, the ovum or both can be contacted with the agent prior to, during and/or subsequently to contact with each other. In one embodiment, the sperm cell and/or the ovum are contacted with the agent prior to contact with each other. In another embodiment, the sperm cell and/or the ovum are contacted with the agent concurrently with being contacted with each other. Preferably, the sperm cell and ovum are human.

The abnormality to which susceptibility is reduced can be any abnormality associated with epigenetic instability. Such abnormalities include, without limitation, cancer, placental enlargement, large offspring syndrome, Angelman syndrome, Prader-Willi syndrome and Beckwith-Wiedeman syndrome. Cancers include, without limitation, a nephroblastoma, a rhabdomyosarcoma, a neuroblastoma and a small cell lung carcinoma.

This invention further provides a method for producing a Dnmt3L⁻ non-human mammalian cell, which method comprises introducing a non-human mammalian Dnmt3L⁻ nucleus into a non-human mammalian enucleated oocyte or zygote under suitable conditions, thereby producing a Dnmt3L⁻ cell. Methods of nuclear introduction into enucleated cells are well known.

This invention further provides a method for producing a non-human mammalian zygote which, upon successful subsequent development in utero, gives rise to a non-human mammal having a reduced susceptibility to an abnormality associated with epigenetic instability, comprising introducing a non-human mammalian Dnmt3L nucleus into a non-human mammalian enucleated oocyte or zygote under suitable conditions (i.e., those permitting creation of a zygote), thereby producing the zygote. This method has particular advantages in reducing the incidence of birth defects associated with cloning animals for agricultural, pharmaceutical and medical (i.e., organ harvesting) uses.

In this method, the non-human mammal can be, for example, a mouse, a rat, a hamster, a pig, a cow, a sheep, a non-human primate, a cat or a dog. The abnormality whose incidence is reduced can be, for example, cancer, placental enlargement, large offspring syndrome, Angelman syndrome, Prader-Willi syndrome and Beckwith-Wiedeman syndrome. Cancers include, without limitation, a nephroblastoma, a rhabdomyosarcoma, a neuroblastoma and a small cell lung carcinoma.

This invention provides a first method for determining the amount of Dnmt3L in a cell comprising (a) contacting the cell's contents with an agent that specifically forms a complex with Dnmt3L, (b) determining the amount of complex formed in step (a), and (c) comparing the amount of complex so determined with a known standard, thereby determining the amount of Dnmt3L in the cell. Agents that specifically form a complex with Dnmt3L include, for example, antibodies.

This invention also provides a second method for determining the amount of Dnmt3L in a cell comprising (a) determining the amount of Dnmt3L-encoding mRNA in the cell and (b) comparing the amount so determined with a known standard permitting the quantitation of Dnmt3L based on a known amount of Dnmt3L-encoding mRNA, thereby determining the amount of Dnmt3L in the cell.

This invention further provides a method for determining whether an agent binds to Dnmt3L comprising contacting the agent with Dnmt3L under suitable conditions, and determining whether the agent forms a complex with the Dnmt3L, the formation of a complex indicating that the agent binds to Dnmt3L. Methods for conducting such binding assays are well known. In one embodiment of the instant assay, the Dnmt3L is part of a fusion protein. In another embodiment, the Dnmt3L is present inside a cell.

This invention provides a first method for determining whether an agent decreases the amount of Dnmt3L in a cell, which method comprises the steps of:

(a) contacting the cell with the agent under suitable conditions;

(b) determining the amount of Dnmt3L in the cell after a suitable period of time; and

(c) comparing the amount of Dnmt3L determined in step (b) with the amount of Dnmt3L in a cell in the absence of the agent, a lower amount of Dnmt3L in the cell contacted with the agent indicating that the agent decreases the amount of Dnmt3L in the cell.

This invention also provides a second method for determining whether an agent decreases the expression of Dnmt3L in a cell, which method comprises the steps of:

(a) contacting the cell with the agent under suitable conditions;

(b) determining the amount of Dnmt3L expression in the cell after a suitable period of time; and

(c) comparing the amount of Dnmt3L expression determined in step (b) with the amount of Dnmt3L expression in a cell in the absence of the agent, a lower amount of Dnmt3L expression in the cell contacted with the agent indicating that the agent decreases the amount of Dnmt3L expression in the cell.

This invention further provides an isolated nucleic acid that specifically hybridizes to Dnmt3L-encoding mRNA. In one embodiment, the nucleic acid is an RNAi molecule, an anti-sense nucleic acid, a ribozyme or a DNAzyme. The instant nucleic acid can be in the form of an expressible nucleic acid, for example.

Finally, this invention provides a composition of matter comprising the instant nucleic acid and a carrier.

This invention will be better understood from the Experimental Details that follow. However, one skilled in the art will readily appreciate that the specific methods and results discussed are merely illustrative of the invention as described more fully in the claims which follow thereafter.

Experimental Details

Introduction

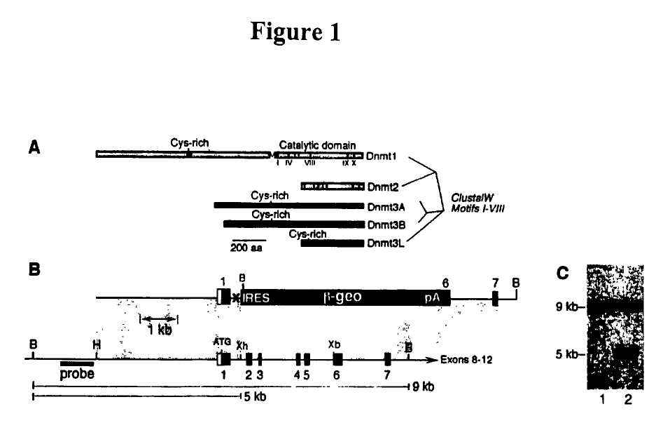

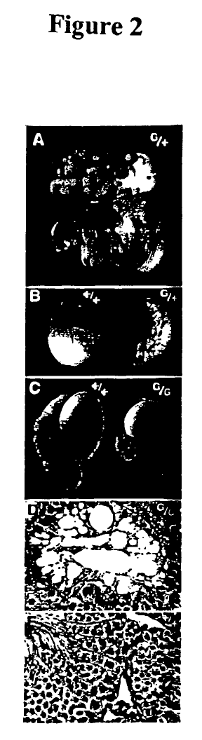

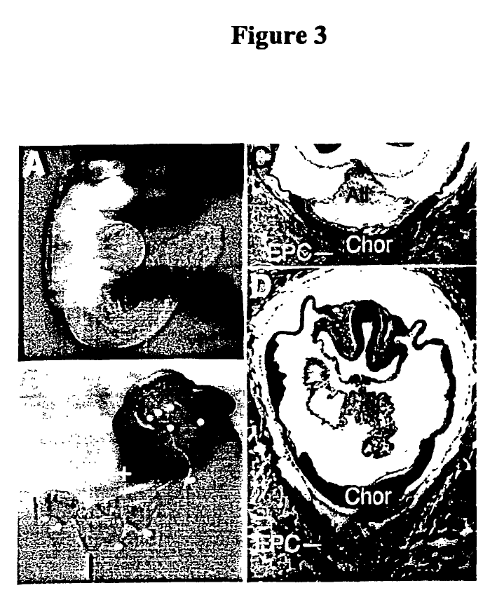

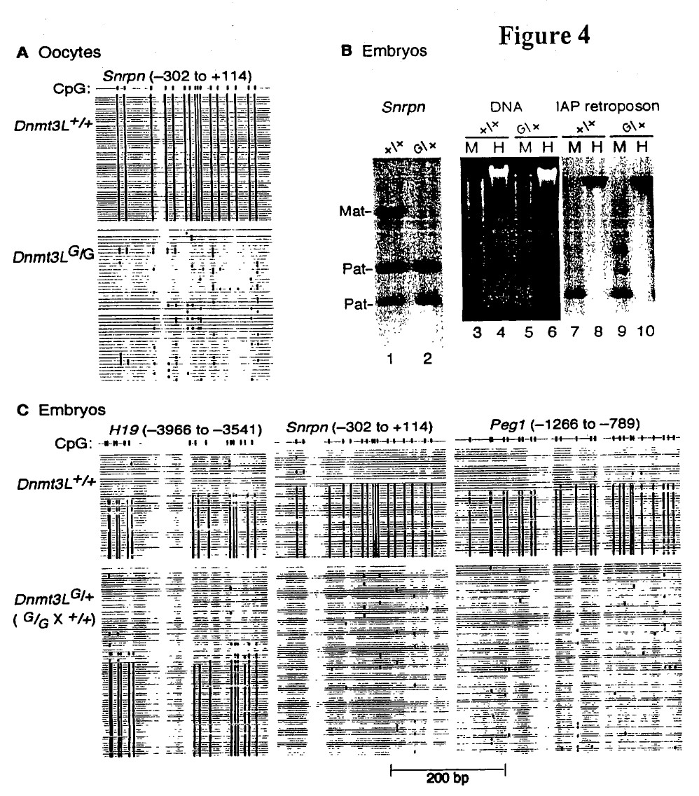

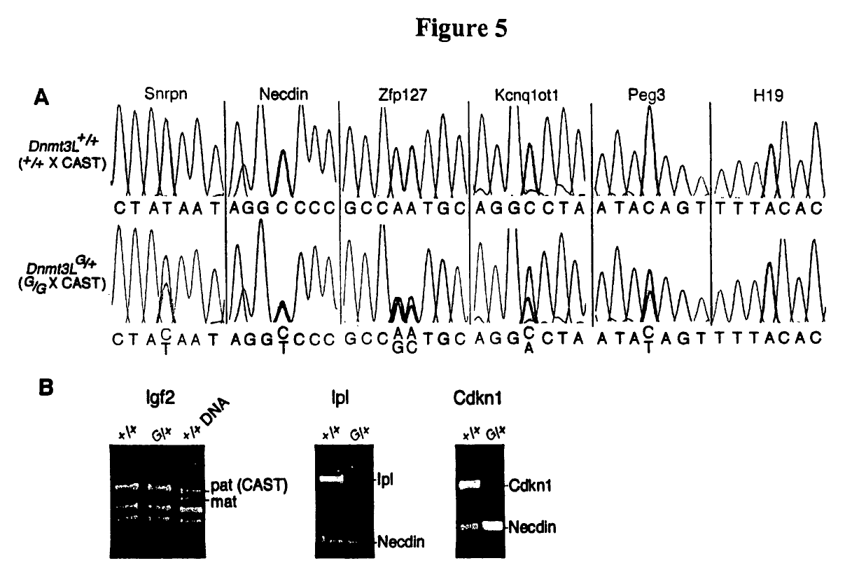

Maternal imprints are established in growing diplotene oocytes and paternal imprints in perinatal prospermatogonia (7-10). In many cases imprinting involves the de novo methylation of regulatory regions of affected genes (reviewed in 4); these methylation marks are maintained throughout development and are only erased and reestablished in the germ line. The DNA methyltransferases and regulatory factors involved in the establishment of imprints in germ cells have not been identified. Dnmt3L (11), whose encoding human mRNA sequence is set forth in GenBank Accession No. NM013369, became a candidate for such an activity based on sequence similarity to Dnmt3A and Dnmt3B, which have been shown to catalyze de novo methylation (12). However, Dnmt3L lacks the sequence motifs shown to be involved in activation of the target cytosine, binding of the methyl donor Sadenosyl L-methionine, and sequence recognition (13). Similarity of Dnmt3L to Dnmt3A and Dnmt3B is largely restricted to a cysteine-rich region of unknown function and regions between catalytic motifs (FIG. 1A).

Methods and Results

Dnmt3L was disrupted by homologous recombination in mouse embryonic stem (ES) cells by means of a deletion-replacement mutation that removed 4 exons and inserted a b-galactosidase-neomycin phosphotransferase (b-geo) fusion gene (14) under the control of the endogenous Dnmt3L promoter (FIGS. 1B, C). The disrupted allele was termed Dnmt3L^{G}. Heterozygous Dnmt3L^{G} mice were of normal phenotype and showed high-level expression of the β-geo marker exclusively in the cell types in which genomic imprints are established (7-10): growing oocytes in adult females and prospermatogonia in perinatal males (FIG. 2A, B). Homozygous animals of both sexes were viable and of normal visible phenotype, but both sexes were sterile. Testes of homozygous Dnmt3L^{G} animals contained normal complements of germ cells at birth but adult testes had severe hypogonadism and a Sertoli-cell-only phenotype (FIG. 2D, E).

Oogenesis was normal in homozygous Dnmt3L^{G} females, but the mutation behaved as a maternal effect lethal in that heterozygous progeny of homozygous females failed to develop past 9.5 days post coitum (dpc). The most notable anatomical abnormalities within the embryo proper were pericardial edema with exencephaly and other neural tube defects (FIG. 3A). These defects together with death at midgestation are common consequences of abnormalities of extraembryonic tissues (15).

In the case of heterozygous progeny of homozygous Dnmt3L^{G} females these included a failure of chorio-allantoic fusion (FIGS. 3C, D), hyperproliferation of secondary trophoblastic giant cells and overgrowth of the chorion, hyperproliferation of yolk sac endoderm, and excess maternal blood in the vicinity of the ectoplacental cone (FIG. 3D). The defects were not due to uterine environment effects, as a similar phenotype was seen when heterozygous progeny of homozygous females were transferred to oviducts of wild type foster females (FIG. 3B).

A role for Dnmt3L in the establishment of genomic imprints was suggested by the specific expression in germ cells at stages where imprints are established, the sequence affinities with known DNA methyltransferases, and the maternal-effect phenotype. Bisulfite genomic sequencing (16) of the differentially methylated region (DMR) of the imprinted and maternally repressed Snrpn gene (17) revealed that the DMR was heavily methylated at all tested sites in DNA of control oocytes but was dramatically undermethylated in oocytes of homozygous Dnmt3L^{G} females (FIG. 4A). DNA blot hybridization after cleavage with the methylation-sensitive restriction endonuclease HhaI was used to determine whether the methylation deficiency present in the oocyte persisted on the maternal allele in progeny derived from crosses to wild type males. Neither allele of Snrpn was detectably methylated in such heterozygous embryos (FIG. 4B). Bisulfite genomic sequencing showed that one-half of the alleles of the imprinted genes H19, Snrpn, and Peg1 were methylated in DNA of control embryos. H19, one of the rare genes whose maternal expression is enforced by methylation of the paternal allele (18, 19), showed normal allele-specific methylation in heterozygous progeny of homozygous females whereas the maternally-imprinted Snrpn and Pegl genes were unmethylated on both alleles (FIG. 4C).

These results showed that Dnmt3L is required for the establishment of maternal methylation imprints during oogenesis, and that a maternal store of Dnmt3L is not required for the maintenance of paternal methylation imprints. Global genome methylation (the large majority of which resides in repeated sequences) was not notably reduced in DNA of heterozygous progeny of homozygous Dnmt3L^{G} mutant females (FIG. 4B), and demethylation imposed by Dnmt3L deficiency during oogenesis was largely restricted to the DMRs of maternally imprinted genes.

The effect of abnormal methylation imprints on imprinted gene expression was tested in embryos derived from crosses of Mus musculus females homozygous for Dnmt3L^{G} to wild type M. m. castaneus (CAST) males or to a strain in which CAST chromosome 7 had been introgressed into a C57BL/6J strain background to improve breeding efficiency. Expressed polymorphisms allowed assignment of parental origin of transcripts. As shown in FIG. 5, Snrpn, Necdin, Zfp127, Kcnqlot1, and Peg3 were transcribed from both alleles in heterozygous Dnmt3L^{G} progeny of homozygous females but only from the paternal allele in control M. musculus X CAST embryos. The paternally methylated and imprinted H19 gene remained maternally expressed, in agreement with the retention of paternal H19 methylation seen in FIG. 4C. Igf2, which is normally maternally repressed (3), retained paternal-specific expression as predicted by unperturbed H19 imprinting (4, 21). Cdkn1 and Ipl were not expressed from either allele, as assessed by reverse transcription-PCR with specific primers and an internal Necdin control (FIG. 5B). This is likely to be due to repression of the maternal allele of Cdkn1 and Ipl as a result of reactivation of the maternal allele of the nearby Kcnqlot1 gene, which is predicted to repress in cis the active allele of other imprinted genes in the cluster (22-24). Development of heterozygous Dnmt3L^{G} embryos is most similar to that of embryos reconstituted from a normal sperm nucleus and a haploid nucleus derived from a non-growing oocyte, which lacks both maternal and paternal imprints (7).

Discussion

Mutations in Dnmt3L and each of the known DNA methyltransferases produce distinct phenotypes. Deletion of the somatic form of Dnmt1 causes global genome demethylation with dysregulation of imprinted genes and ectopic X chromosome inactivation (25, 26), while deletion of the oocyte-specific form of Dnmt1 causes a pure maternal effect phenotype in which one-half of the normally silent alleles of certain imprinted genes are demethylated and reactivated in heterozygous progeny of homozygous females (27). Mutations in Dnmt3B prevent the methylation of specific types of pericentric satellite DNA and cause the human immunodeficiency and chromosome instability disease known as ICF syndrome (28).

While demethylation has not been reported to occur in Dnmt3A-deficient cells, Dnmt3A-Dnmt3B double mutant ES cells have been reported to undergo global genome demethylation and a loss of the ability to methylate newly-integrated retroviral DNA (12). Dnmt3L is required specifically for the establishment of genomic imprints but dispensable for their propagation, and Dnmt3L is the only gene known to be essential for the de novo methylation of single-copy DNA sequences.

The results of this and prior studies (27) confirm that the methylation of single copy sequences and repeated sequences are independently regulated. The sequence of Dnmt3L suggests that the protein is likely to function not directly as a DNA methyltransferase but as a regulator of methylation at imprinted loci.

### REFERENCES

1. J. McGrath, D. Solter, Cell 37, 179 (1984).

2. S. C. Barton, M. A. Surani, M. L. Norris, Nature 311, 374 (1984).

3. A. Efstratiadis, Curr. Op. Genet. Dev. 4, 265 (1994).

4. S. M. Tilghman, Cell 96, 185 (1999).

5. B. Tycko, Probl. Cell. Differ. 25, 133 (1999).

6. D. Humpherys et al., Science 293, 95 (2001).

7. T. Kono, Y. Obata, T. Yoshimzu, T. Nakahara, J. Carroll, Nat Genet. 13, 91 (1996).

8. T. L. Davis, G. J. Yang, J. R. McCarrey, M. S. Bartolomei, Hum. Mol. Genet. 9, 2885 (2000).

9. T. Ueda et al., Genes Cells 5, 649 (2000).

10. R. Stoger et al., Cell 73, 61 (1993).

11. U. Aapola et al., Genomics 65, 293 (2000).

12. M. Okano, S. Xie, E. Li, Nature Genet. 19, 219 (1998).

13. S. Klimasauskas, S. Kumar, R. J. Roberts, X. Cheng, Cell 76, 357 (1994).

14. C. L. Halbert, I. E. Alexander, G. M. Wolgamot, A. D. Miller, J. Virol. 69, 1473 (1995).

15. A. J. Copp, Trends Genet. 11, 87 (1995).

16. S. J. Clark, J. Harrison, C. L. Paul, M. Frommer, Nucleic Acids Res. 22, 2990 (1994).

17. C. C. Glenn, K. A. Porter, M. T. Jong, R. D. Nicholls, D. J. Driscoll, Hum. Mol. Genet. 2, 2001 (1993).

18. M. S. Bartolomei, A. L. Webber, M. E. Brunkow, S. M. Tilghman, Genes Dev. 7, 1663 (1993).

19. A. C. Ferguson-Smith, H. Sasaki, B. M. Cattanach, M. A. Surani, Nature 362, 751 (1993).

20. C. P. Walsh, J. R. Chaillet, T. H. Bestor, Nature Genet. 20, 116 (1998).

21. P. E. Szabo, J. R. Mann, Genes Dev. 9, 3097 (1995).

22. S. Horike et al., Hum. Mol. Genet. 9, 2075 (2000).

23. N. J. Smilinich et. al., Proc. Nat. Acad. Sci. USA 96, 8064 (1999).

24. M. A. Cleary, C. D. van Raamsdonk, J. Levorse, B. Zheng, A. Bradley, S. M. Tilghman, Nature Genet. 29, 78 (2001).

25. E. Li, T. H. Bestor, R. Jaenisch, Cell 69, 915 (1992).

26. T. H. Bestor, Hum. Mol. Genet. 9, 2395 (2000).

27. C. Y. Howell et al., Cell 104, 829 (2001).

28. G. -L. Xu et al., Nature 402, 187 (1999).

What is claimed is:

1. A method for reducing the amount of Dnmt3L in a cell comprising contacting the cell under suitable conditions with an agent that specifically inhibits the expression of Dnmt3L in the cell, thereby reducing the amount of Dnmt3L in the cell.

2. The method of claim 1, wherein the agent is a nucleic acid.

3. The method of claim 2, wherein the nucleic acid is DNA.

4. The method of claim 2, wherein the nucleic acid is RNA.

5. The method of claim 2, wherein the nucleic acid is selected from the group consisting of an RNAi molecule, an antisense nucleic acid, a ribozyme and a DNAzyme.

6. The method of claim 1, wherein the cell is a human cell.

7. The method of claim 1, wherein the cell is a stem cell.

8. The method of claim 1, wherein the cell is an embryonic cell.

9. The method of claim 1, wherein the cell is a gamete.

10. The method of claim 1, wherein the expression of Dnmt3L in the cell is eliminated.

11. A method for obtaining a cell having a reduced amount of Dnmt3L comprising isolating a cell from a transgqnic non-human mammal whose cells contain a reduced amount of Dnmt3L.

12. The method of claim 11, wherein the cell is a stem cell.

13. The method of claim 11, wherein the cell is an embryonic cell.

14. The method of claim 11, wherein the cell is a gamete.

15. The method of claim 11, wherein no Dnmt3L is expressed in the cell.

16. The method of claim 11, wherein the transgenic non-human mammal's cells contain no Dnmt3L.

17. An isolated cell having a reduced amount of Dnmt3L.

18. The cell of claim 17, wherein the cell is selected from the group consisting of a hepatocyte, a neuron, a cardiac cell, a stem cell, an embryonic cell and a skin cell.

19. An isolated population of cells having a reduced amount of Dnmt3L.

20. The population of cells of claim 19, wherein the cells are selected from the group consisting of hepatocytes, neurons, cardiac cells, stem cells, embryonic cells and skin cells.

21. A tissue comprising cells having a reduced amount of Dnmt3L.

22. The tissue of claim 21, wherein the tissue is selected from the group consisting of hepatic tissue, neuronal tissue, cardiac tissue and skin tissue.

23. An organ comprising cells having a reduced amount of Dnmt3L.

24. The organ of claim 23, wherein the organ is selected from the group consisting of a liver, a heart and skin.

25. A non-human transgenic mammal comprising cells having a reduced amount of Dnmt3L.

26. The mammal of claim 25, wherein the cells having the reduced amount of Dnmt3L have no Dnmt3L.

27. The mammal of claim 25, wherein the mammal is a mouse, a rat, a hamster, a pig, a cow, a sheep, a non-human primate, a cat or a dog.

28. A method for producing a mammalian zygote in vitro which, upon successful subsequent development in utero, gives rise to a mammal having a reduced susceptibility to an abnormality associated with epigenetic instability, comprising contacting a mammalian sperm cell and a mammalian ovum in vitro under conditions permitting fertilization of the ovum by the sperm, wherein the sperm cell, the ovum or both are contacted with an agent that reduces the amount of Dnmt3L therein, thereby producing the mammalian zygote.

29. The method of claim 28, wherein the sperm cell and/or the ovum are contacted with the agent prior to contact with each other.

30. The method of claim 28, wherein the sperm cell and/or the ovum are contacted with the agent concurrently with being contacted with each other.

31. The method of claim 28, wherein the sperm cell and ovum are human.

32. The method of claim 28, wherein the abnormality is selected from the group consisting of cancer, placental enlargement, large offspring syndrome, Angelman syndrome, Prader-Willi syndrome and Beckwith-Wiedeman syndrome.

33. The method of claim 32, wherein the cancer is selected from the group consisting of a nephroblastoma, a rhabdomyosarcoma, a neuroblastoma and a small cell lung carcinoma.

34. A method for producing a Dnmt3L⁻ non-human mammalian cell, which method comprises introducing a non-human mammalian Dnmt3L nucleus into a non-human mammalian enucleated oocyte or zygote under suitable conditions, thereby producing a Dnmt3L⁻ cell.

35. A method for producing a non-human mammalian zygote which, upon successful subsequent development in utero, gives rise to a non-human mammal having a reduced susceptibility to an abnormality associated with epigenetic instability, comprising introducing a non-human mammalian Dnmt3L⁻ nucleus into a non-human mammalian enucleated oocyte or zygote under suitable conditions, thereby producing the zygote.

36. The mammal of claim 35, wherein the non-human mammal is a mouse, a rat, a hamster, a pig, a cow, a sheep, a non-human primate, a cat or a dog.

37. The method of claim 35, wherein the abnormality is selected from the group consisting of cancer, placental enlargement, large offspring syndrome, Angelman syndrome, Prader-Willi syndrome and Beckwith-Wiedeman syndrome.

38. The method of claim 37, wherein the cancer is selected from the group consisting of a nephroblastoma, a rhabdomyosarcoma, a neuroblastoma and a small cell lung carcinoma.

39. A method for determining the amount of Dnmt3L in a cell comprising (a) contacting the cell's contents with an agent that specifically forms a complex with Dnmt3L, (b) determining the amount of complex formed in step (a), and (c) comparing the amount of complex so determined with a known standard, thereby determining the amount of Dnmt3L in the cell.

40. A method for determining the amount of Dnmt3L in a cell comprising (a) determining the amount of Dnmt3L-encoding mRNA in the cell and (b) comparing the amount so determined with a known standard permitting the quantitation of Dnmt3L based on a known amount of Dnmt3L-encoding mRNA, thereby determining the amount of Dnmt3L in the cell.

41. A method for determining whether an agent binds to Dnmt3L comprising contacting the agent with Dnmt3L under suitable conditions, and determining whether the agent forms a complex with the Dnmt3L, the formation of a complex indicating that the agent binds to Dnmt3L.

42. The method of claim 41, wherein the Dnmt3L is part of a fusion protein.

43. The method of claim 41, wherein the Dnmt3L is present inside a cell.

44. A method for determining whether an agent decreases the amount of Dnmt3L in a cell, which method comprises the steps of:
(a) contacting the cell with the agent under suitable conditions;
(b) determining the amount of Dnmt3L in the cell after a suitable period of time; and
(c) comparing the amount of Dnmt3L determined in step (b) with the amount of Dnmt3L in a cell in the absence of the agent, a lower amount of Dnmt3L in the cell contacted with the agent indicating that the agent decreases the amount of Dnmt3L in the cell.

45. A method for determining whether an agent decreases the expression of Dnmt3L in a cell, which method comprises the steps of:
(a) contacting the cell with the agent under suitable conditions;
(b) determining the amount of Dnmt3L expression in the cell after a suitable period of time; and
(c) comparing the amount of Dnmt3L expression determined in step (b) with the amount of Dnmt3L expression in a cell in the absence of the agent, a lower amount of Dnmt3L expression in the cell contacted with the agent indicating that the agent decreases the amount of Dnmt3L expression in the cell.

46. An isolated nucleic acid that specifically hybridizes to Dnmt3L-encoding mRNA.

47. The nucleic acid of claim 46, wherein the nucleic acid is selected from the group consisting of an RNAi molecule, an anti-sense nucleic acid, a ribozyme and a DNAzyme.

48. A composition of matter comprising the nucleic acid of claim 46 and a carrier.
